(19) United States
(12) Patent Application Publication
(10) Pub. No.: US 2024/0315859 A1
(43) Pub. Date: Sep. 26, 2024

(54) ENDOVASCULAR IMPLANT POSITIONING APPARATUS WITH FUNCTIONALLY MODIFIED WIRE

(71) Applicant: BARD PERIPHERAL VASCULAR, INC., Franklin Lakes, NJ (US)

(72) Inventor: Ziv J. Haskal, Charlottesville, VA (US)

(21) Appl. No.: 18/575,638

(22) PCT Filed: Jun. 30, 2021
(86) PCT No.: PCT/US2021/039772

§ 371 Date: Dec. 29, 2023

(51) Int. Cl. A61F 2/962; A61F 2/954

(52) U.S. Cl. CPC A61F 2/962; A61F 2/954

(57) ABSTRACT

An apparatus is for positioning an endovascular implant, such as a stent to assist in maintaining vessel patency, adjacent a junction at a branch from a vessel. A sheath includes a first lumen and a second lumen. The first lumen is adapted for receiving a catheter including the endovascular implant for positioning within the branch. An at least partially radiopaque wire is located in the second lumen. The at least partially radiopaque wire includes a pre-shaped portion, such as a bend, for locating at the junction for use in guiding the stent into position within the branch. Related methods are also disclosed.

### BACKGROUND OF THE INVENTION

Several disease states and anatomies involve placing an endovascular implant, such as for example a stent for maintaining vessel patency, with high precision at the edge of a confluence, or branch. Precise placement is desirable to ensure full coverage of the intended area and, at the same time, prevent complications from blockage (or “jailing” and excessively covering) the adjacent vessel or lumen. Examples include atherosclerosis, venous turbulence, or external anatomic compression, which commonly lead to areas of flow separation, stenosis, intimal hyperplasia and/or plaque formation. Junctions and branch points are exposed to flow turbulence, which can commonly result in stenoses and occlusions at those points. Effective and durable therapeutic implant placement must precisely cover these areas, flush with or slightly extending into the main or source vessel, or else narrow segments remain. At the same time, excessive protrusion into the adjacent vessel or lumen must be avoided to prevent secondary blockages and allow for future possible interventions.

Current modalities to achieve precise implant placement at a confluence involve imaging techniques in which the orientation and view of the X-ray beam is changed several times so that the operator can try to assemble a mental three-dimensional understanding of the relevant anatomic challenge and the orientation of the vessels and ducts. Not only is this time consuming, but it requires additional use of fluoroscopy and radiation exposure. The outcome depends upon the consistent high level of operator comprehension and skill.

Furthermore, the procedure requires additional use of iodinated contrast injection, increasing risks of contrast induced complications, and may be near impossible to precisely define due to excessive patient body habitus limiting fluoroscopic visibility. The procedure also does not account for target motion with respiration during imaging, and may not be addressable by fluoroscopy alone, as representative orthogonal views cannot be obtained with conventional fluoroscopy. The technical challenges are even increased when the stent or covered stent is placed in the venous system where the flow of blood and contrast media often is retrograde.

Accordingly, a need is identified for a solution to the foregoing problems, and perhaps others that have yet to be discovered or understood. The solution should provide reliable and accurate positioning of an endovascular implant in a repeatable manner, and without significant cost or complexity in terms of the implementation.

### SUMMARY OF THE INVENTION

An object of the disclosure is to provide a positioning apparatus for an endovascular implant by way of which a wire having a functionally modified portion with a predetermined shape, such as a bend, which may be advanced into an adjacent vessel at the junction point and positioned against the junction wall, visibly defining the location for the implant to be deployed. Under fluoroscopy, the wire thus provides an easily visualized and perceptible manner to achieve implant (e.g., stent, covered stent, stent graft, etc.) positioning, independent of contrast injection or respiratory or motion. The apparatus may remain deployed to allow continued positioning feedback during final stent positioning and stent release. This may reduce procedure time, radiation exposure, use of contrast media, while concomitantly improving placement accuracy and helping to ensure consistent operator success, all without significant increases in cost or complexity.

According to one aspect of the disclosure, an apparatus for positioning an endovascular implant adjacent a junction including a branch from a vessel is provided. The apparatus comprises a sheath including a first lumen and a second lumen. The first lumen is adapted for receiving a catheter adapted for positioning the endovascular implant within the branch. An at least partially radiopaque wire is located in the second lumen. The at least partially radiopaque wire includes a pre-shaped portion for positioning at the junction for use in guiding the endovascular implant into position within the branch.

In one embodiment, the first lumen has a larger diameter than the second lumen. The at least partially radiopaque wire may comprise metal, or a shape memory material. An end portion or extension of the radiopaque wire distal of the pre-shaped portion is adapted for engaging an adjacent wall of the vessel at the branch.

An opening of the second lumen may be provided at a distal end of the sheath. Alternatively, or additionally, an opening of the second lumen may be proximal of a distal end of the sheath. For example, the opening may be a lateral slot to allow the wire to exit the sheath.

The pre-shaped portion may comprise a bend in the at least partially radiopaque wire proximal of a distal end of the at least partially radiopaque wire. The bend may be adapted to engage an end of the endovascular implant (leading or trailing, depending on the situation).

A further aspect of the disclosure pertains to an apparatus for positioning an endovascular implant adjacent a junction including a branch from a vessel. The apparatus comprises a sheath including a first lumen and a second lumen. A catheter is at least partially positioned in the first lumen, the catheter adapted for positioning the endovascular implant within the branch. An at least partially radiopaque wire is located in the second lumen. The at least partially radiopaque wire includes a preshaped portion for positioning at the junction for use in guiding the endovascular implant into position within the branch.

In one embodiment, the first lumen has a larger diameter than the second lumen. The at least partially radiopaque wire comprises metal, and may comprise a shape memory material. An extension of the at least partially radiopaque wire is adapted for engaging an adjacent wall of the vessel at the branch.

An opening of the second lumen may be located at a distal end of the sheath. Alternatively, or additionally, an opening of the second lumen is proximal of a distal end of the sheath. For example, the opening may be a lateral slot to allow the wire to exit the sheath.

The pre-shaped portion may comprise a bend in the at least partially radiopaque wire proximal of a distal end of the at least partially radiopaque wire. The bend may be adapted to engage an end of the endovascular implant.

Still a further aspect of the disclosure pertains to an apparatus for positioning an endovascular implant adjacent a junction including a branch from a vessel using a sheath including a first lumen and a second lumen, the first lumen adapted for receiving a catheter adapted for positioning the endovascular implant within the branch. The apparatus comprises an at least partially radiopaque wire adapted to be located in the second lumen. The at least partially radiopaque wire includes a preshaped portion adapted for positioning at the junction for use in guiding the endovascular implant into position within the branch.

In one embodiment, the at least partially radiopaque wire comprises metal. The at least partially radiopaque wire may comprise a shape memory material.

An extension of the at least partially radiopaque wire may be adapted for engaging an adjacent wall of the vessel at the branch. The pre-shaped portion may comprise a bend in the at least partially radiopaque wire proximal of a distal end of the at least partially radiopaque wire. The bend may be adapted to engage an end of the endovascular implant.

Yet a further aspect of the disclosure pertains to a method of positioning an endovascular implant in a branch from a vessel. The method comprises providing a sheath including a first lumen, and extending an at least partially radiopaque wire from the first lumen to locate a preshaped portion of the at least partially radiopaque wire adjacent the branch for use in guiding the endovascular implant into position within the branch. The method may further include the step of providing the sheath with a second lumen, and advancing a catheter including the endovascular implant via the second lumen into the branch. Still further, the method may comprise engaging the endovascular implant with the pre-shaped portion of the wire, which may comprise a bend.

### BRIEF DESCRIPTION OF THE DRAWINGS

The above and further advantages of the present disclosure may be better understood by referring to the following description in conjunction with the accompanying drawings in which:

FIG. 1 is a side cross-sectional view of a sheath forming part of the positioning apparatus according to one aspect of the disclosure;

FIGS. 1A and 1B are enlarged, partially cutaway views of different end portions or tips of the sheath of FIG. 1;

FIG. 2 is a side view of a wire forming part of the positioning apparatus according to one aspect of the disclosure;

FIG. 2A is an enlarged, partially cutaway view of a portion of the wire of FIG. 2;

FIG. 3 is a view of one example of a delivery device for an endovascular implant;

FIG. 3A is a cross-sectional view of an expandable element associated with the delivery device of FIG. 3;

FIGS. 4, 5, 6, 7, and 8 are schematic views showing a possible use of the positioning apparatus; and

FIGS. 9, 10, and 11 illustrate other possible uses of the positioning apparatus.

The dimensions of some of the elements may be exaggerated relative to other elements for clarity or several physical components may be included in one functional block or element. Further, sometimes reference numerals may be repeated among the drawings to indicate corresponding or analogous elements. Moreover, some of the parts depicted in the drawings may be combined into a single function.

### DETAILED DESCRIPTION

In the following detailed description, numerous specific details are set forth to provide a thorough understanding of the presently disclosed invention(s). The disclosed embodiments may be practiced without these specific details. In other instances, well-known methods, procedures, components, or structures may not have been described in detail so as not to obscure the disclosed inventive concepts.

The invention of this disclosure is not limited in its application to the details of construction and the arrangement of the components set forth in the following description or illustrated in the drawings. The inventive concepts disclosed are capable of other embodiments or of being practiced or carried out in various ways. Also, it is to be understood that the phraseology and terminology employed herein are for the purpose of description and should not be regarded as limiting.

Certain features of the disclosed embodiments that are, for clarity, described in the context of separate embodiments, may also be provided in combination in a single embodiment. Conversely, various features of the invention that are, for brevity, described in the context of a single embodiment, may also be provided separately or in any suitable sub-combination.

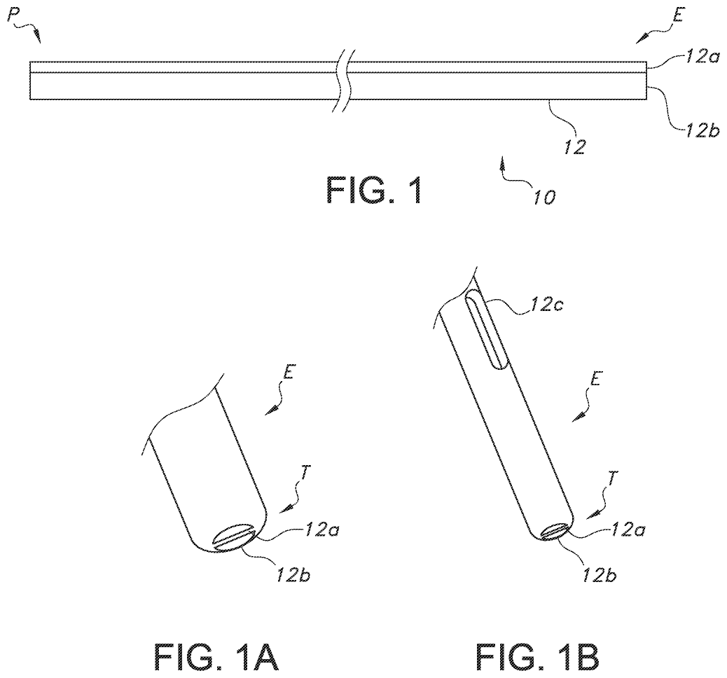

With reference to FIGS. 1-3, a positioning apparatus 10 for an endovascular Implant according to a first aspect of the disclosure is shown. Referring first to the cross-sectional view of FIG. 1, the apparatus 10 includes an elongated, tubular sheath 12. The sheath 12 includes first and second adjacent lumens 12a, 12b, which may be arranged in a side-by-side manner.

The lumens 12a, 12b may be fully coextensive and have different diameters or sizes. For example, one lumen 12a may be substantially smaller than the other lumen 12b, as perhaps best understood by the enlarged showing of the distal end portion E in FIG. 1A. In other words, the second lumen 12b is larger than the first lumen 12a, which may be oriented posteriorly. Additionally, or alternatively, one of the lumens, such as the smaller lumen 12a, may also communicate with a lateral opening, such as an elongated slot 12c, formed along the distal end portion of the sheath 12, the purpose of which will be understood upon reviewing the following description.

The distal end portion E of the sheath 12 may have an atraumatic or rounded tip T, as shown. This tip T is adapted to be located at or adjacent to a treatment location in a vessel and, in particular, at a junction with a branch in the vessel, as will be outlined further in the following description. This may be achieved while a proximal end P of the sheath 12 remains external to the vessel and associated body for manipulation by a clinician.

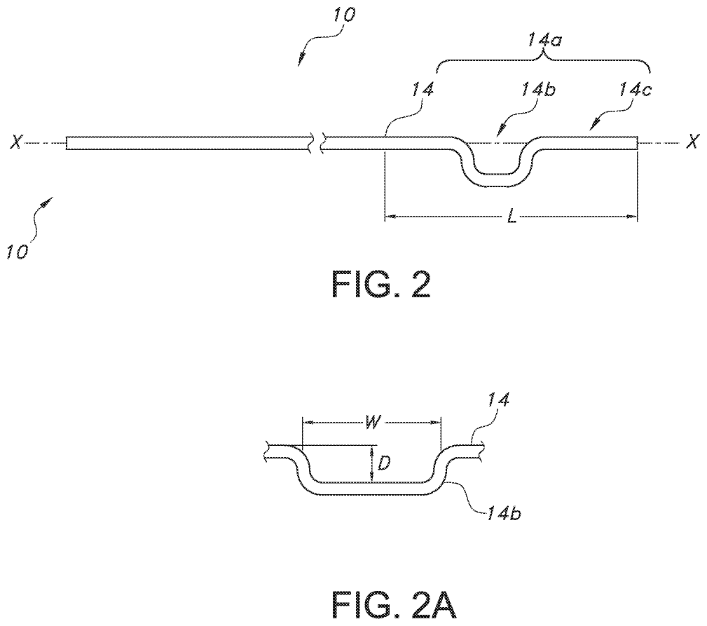

With reference to FIG. 2, the positioning apparatus 10 further includes a wire 14. The wire 14 is adapted to be positioned in and extend through the first, smaller lumen 12a of the sheath 12, and is greater in length so that a distal end portion 14a having a length L extends therefrom. As illustrated, this distal end portion 14a includes a functionally modified, or pre-shaped portion, such as a bend 14b having a depth D relative to a longitudinal axis X of the wire and a width W. Consequently, the bend 14b may have a U-shape when viewed from the side, with two curved portions forming a U-shape, with the wire 14 remaining in a common plane along the bend (but could also be a compound bend).

The distal end portion 14a of the wire 14 also includes an extension 14c. This extension 14c has a shape adapted for engaging a portion of a vessel in which it is positioned. The extension 14c may be part of the bend 14b or formed independently from it.

To allow for identification under imaging, and fluoroscopy in particular, the wire 14 may at least partially comprise a radiopaque material. In particular, the distal end portion 14a may be made of a radiopaque material, such as metal wire. A shape memory material (such as Nitinol, in one particular example) may also be used for reasons outlined further herein, but other materials could be used instead, as long as such allow for the identification outside of the associated body using imaging techniques.

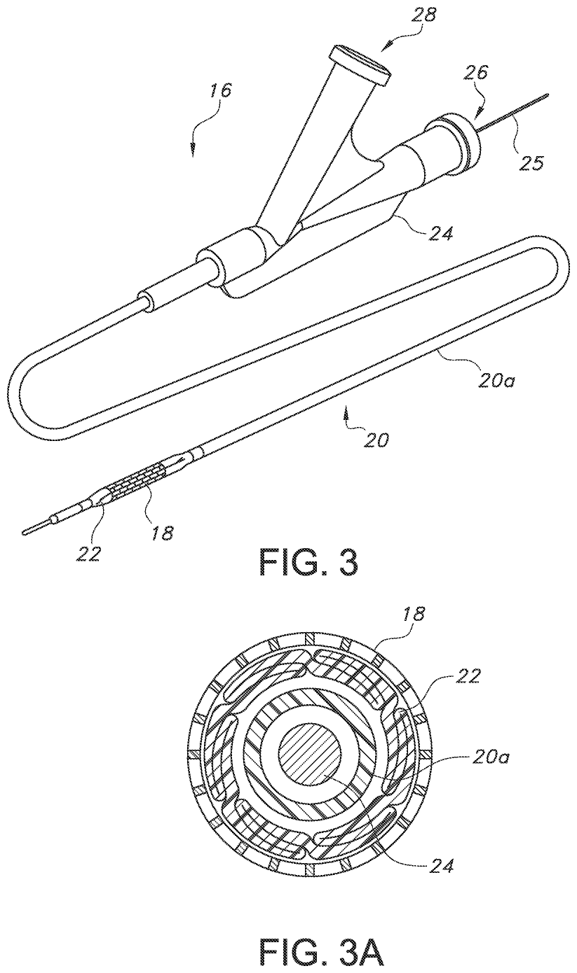

FIG. 3 illustrates that the apparatus 10 may also include or be adapted for use in connection with a delivery device 16 for an endovascular implant, such as a stent 18. The delivery device 16 may include a catheter 20 having an expandable element, such as a balloon 22 (shown folded in FIG. 3A). The catheter 20 may include a conventional hub 24 for manipulation, as well as for guidance via a guidewire 25 inserted through a corresponding guidewire port 26 and inflation of the balloon 22 via an inflation port 28 therein for communicating with corresponding lumens in a shaft 20a of the catheter 20. The catheter 20 may be sized and shaped for delivery to a treatment location via the second lumen 12b of the sheath 12, but could also be for delivery independent of the sheath 12, depending on the circumstances of a given procedure.

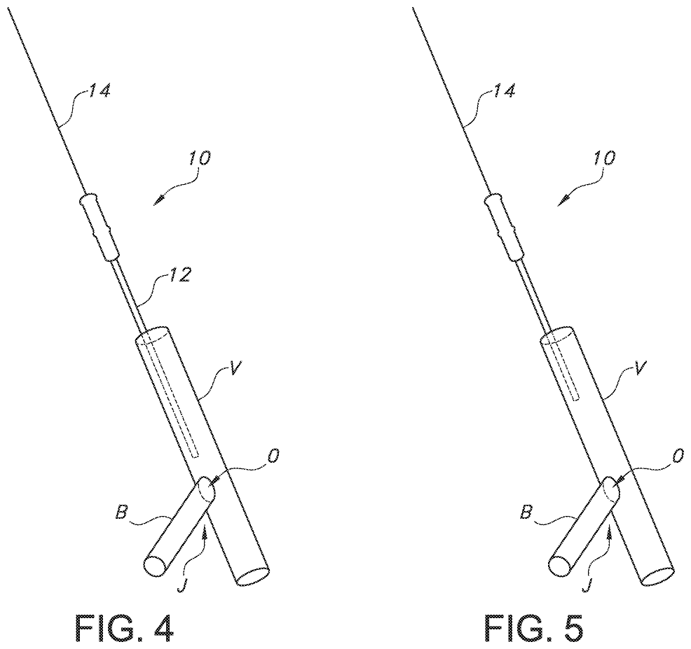

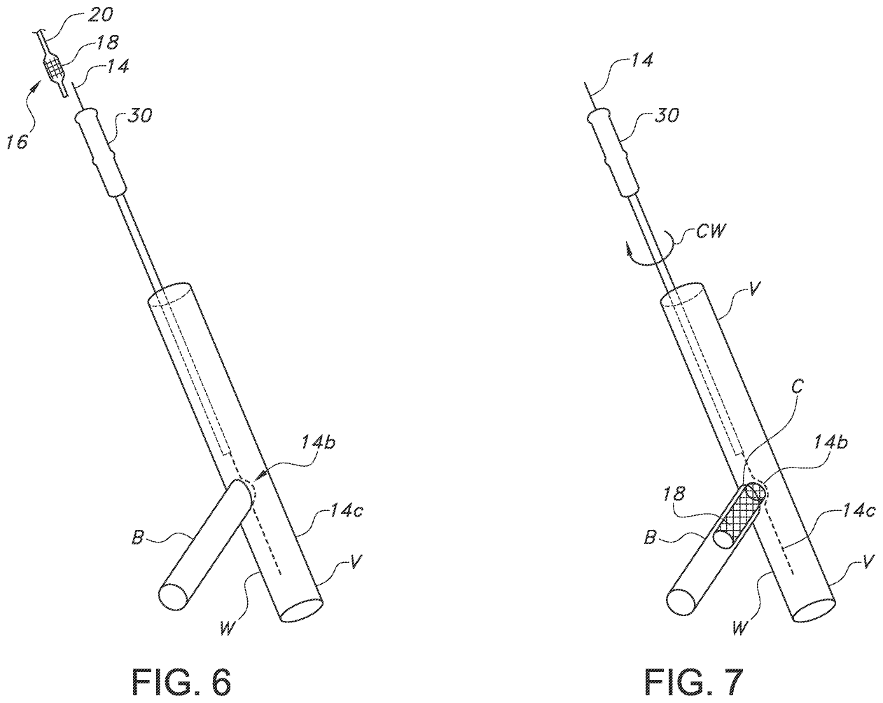

Turning to the schematic views of FIGS. 4, 5, 6, and 7, a possible use of the positioning apparatus 10 is illustrated. Turning first to FIG. 4, a vessel V having a branch B corresponding to a treatment location and forming a junction J is shown. The sheath 12 of the apparatus 10 may be advanced to a location proximal of the branch B, as shown. The wire 14 may then be advanced through the first, smaller lumen 12a and alight therefrom (either via the open end of the lumen 12a or the side opening or slot 12c, as shown in FIGS. 6 and 7).

As can be further understood from FIGS. 6 and 7, the advance is such that the functionally modified portion of the wire 14, or preshaped bend 14b, is located at the ostium O of the branch B. The sheath 12 or wire 14 may be rotated (note arrow CW for clockwise, but counterclockwise rotation is of course possible) as necessary to orient the recess formed by the bend 14b such that it faces the ostium O. An optional hub 30 may be provided to facilitate the relative movement by the clinician from external to the associated body of the patient.

The delivery device 16, or catheter 20 including the stent 18 may then be delivered to the branch, such as via the second larger lumen 12b of sheath 12, and deployed by inflating the balloon 22. As can be appreciated from FIGS. 6 and 7, the positioning of the bend 14b provides visual guidance about the adjacent border of the junction J to facilitate stent placement (flush or protruding, as outlined further below). This allows the clinician to understand precisely and without undue guesswork the location of the ostium O and the positioning of the stent 18 during deployment, thus ensuring improved accuracy and avoiding the above-referenced issues. Moreover, the apparatus 10 may remain deployed to allow continued positioning feedback during final implant/stent 18 positioning and release, and may even be redeployed if necessary (or perhaps even repositioned for placement of a second implant).

The width W and, in particular, the depth D of the bend 14b may be selected by the clinician depending on a desired outcome (i.e., whether the stent 18 or other implant should protrude from the branch a small, but repeatable distance upon deployment, in which case the bend 14b may serve as a locator for the corresponding end of the stent 18). This allows for the more precise positioning of the stent 18 such that it may partially project from the branch B into the vessel V (note distance C of extension), while potentially avoiding the problem of “jailing,” or excessive covering, of the vessel in a manner that impedes flow. Again, the apparatus 10 may also remain deployed to allow continued positioning feedback during final stent positioning and stent release, or redeployed if necessary.

Furthermore, the shaped extension 14c of the wire 14 may align with and contact a wall W of the vessel V adjacent the ostium O of the branch B. This contact by the shaped extension 14c and the resulting visibility creates a reference location, helping to ensure that the depth D at the bend 14b is representative of the desired extension of the stent 18 from the branch B.

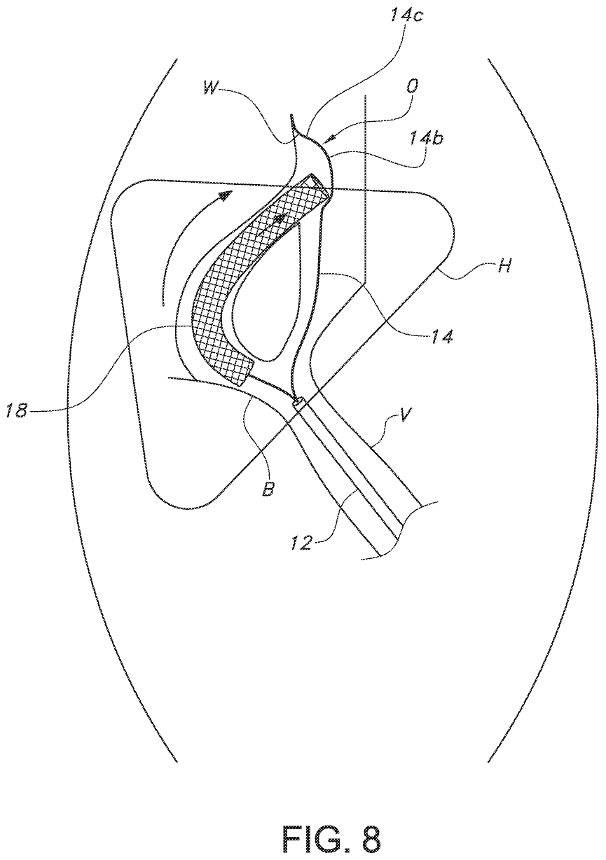

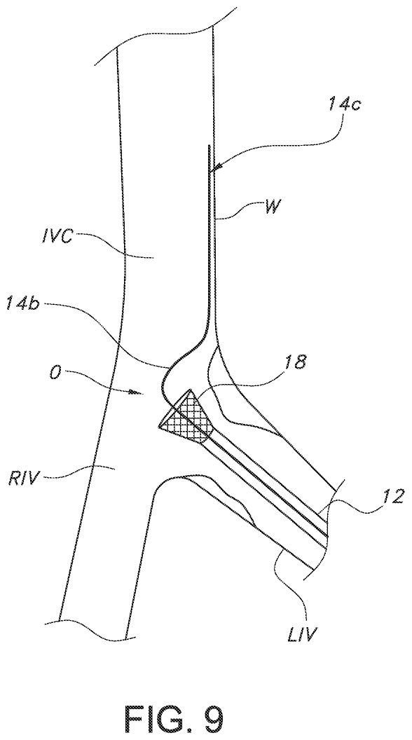

Turning to FIG. 8, an alternative approach is illustrated. Here, the sheath 12 may be positioned in a supply vessel V, such as associated with the liver H. The wire 14 is advanced past the ostium O. Once in position with the pre-shaped portion or bend 14b serving the positioning function, with the extension 14c engaging the adjacent wall W, the stent 18 installed in a branch B. The difference of course is that it is the forward end of the stent 18 projecting into the vessel V from the branch B that may engage the wire bend 14b in this arrangement.

FIGS. 9-11 illustrate possible uses of the apparatus 10 according to the disclosure. With reference to FIG. 8, a condition known as May-Thurner Syndrome results when the left iliac vein LIV is compressed by the right iliac artery (not shown). This condition raises a concern over deep vein thrombosis, which can have deleterious consequences. Improving or restoring patentcy to the left iliac vein LIV via stenting is a commonly proposed treatment for addressing this concern.

As illustrated, the apparatus 10 of the disclosure may be used to ensure the proper positioning of the stent 18 at the ostium O of the left iliac vein LIV, without interfering with the blood flow in the adjacent right iliac vein RIV. Specifically, the sheath 12 may be positioned adjacent the ostium O of the left iliac vein LIV. The wire 14 is then deployed such that the bend 14b is located at the ostium O for engaging the corresponding end of the stent 18 when deployed, and providing visual guidance about the adjacent border of the junction to facilitate stent placement (flush or otherwise). The extension 14c of the wire 14 may also contact the adjacent wall W of the inferior vena cava IVC to ensure the desired location for the bend 14b is achieved. The stent 18 or other implant may then be deployed as described above, without the need to cross the right iliac vein RIV. Moreover, the desired precise extension distance (usually about 2 millimeters) for the implant (stent 18) may be reliably achieved.

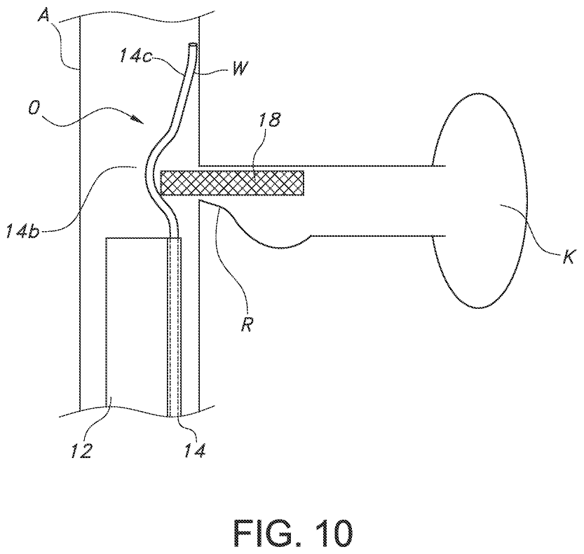

FIG. 10 illustrates a possible use in inserting an implant, such as a stent 18, in the renal artery R supplying blood flow to an associated kidney K to treat atherosclerosis, for example, or otherwise restore patentcy. The sheath 12 may be positioned in the aorta A and the wire 14 positioned with the extension 14c in contact with the wall W adjacent the ostium O of the renal artery R. The bend 14b is located to provide an imaging target for deploying a stent 18 in the renal artery R, as well as a physical location for engagement to avoid extending the stent an undesired amount into the aorta A.

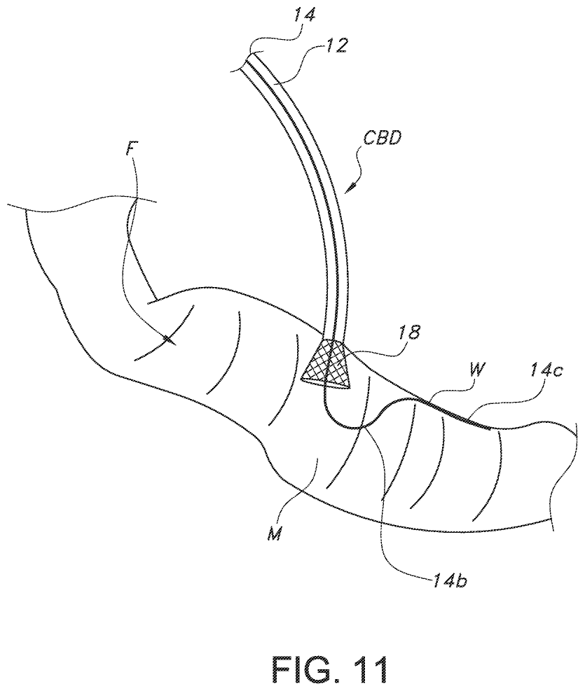

Use of the apparatus 10 for positioning an implant at other locations is also possible. FIG. 11 illustrates a stent 18 installed in the common bile duct CBD associated with the duodenum M. The sheath 12 may be positioned in the common bile duct CBD, with the wire 14 positioned in the duodenum M and the extension contacting an adjacent wall W. The positioning of the bend 14b is such that the proper positioning of the stent 18 may be achieved for deployment without interfering with flow F through the duodenum M.

Other examples of the need for precision stent placement in veins and arteries include, but are not limited to: (i) cephalic arch stenoses in hemodialysis patients with ipsilateral arteriovenous fistulae or grafts; (ii) visceral fenestrations in complex EVAR (e.g. branches, chimneys); (iii) iliac artery lesions close to the internal iliac artery or at the aorto/iliac bifurcation; (iv) placement of a covered stent/stent graft for the creation of a trans jugular intrahepatic portosystemic shunt (TIPS, where is it of high importance to land the trailing end of the stent right at the IVC junction with no more than 2 mm protruding into the inferior vena cava (IVC)).

The materials used for the sheath 12 and wire 14 may vary. Again, the distal portion 14a of the wire 14 should be visible outside of the body, and thus may be at least partially radiopaque (such as along the entire distal end portion 14a). This typically involves the use of metals (solid or coatings), but other types of non-metal (e.g., polymeric) radiopaque materials exist.

As can be further appreciated by a skilled artisan, the precise shape of the bend 14b or bulge may depend on the particular anatomical location at which it is to be used. In the U-shaped example shown, a typical value for the depth D could be about 2-3 millimeters, and the width W 10-20 millimeters. However, these dimensions may be varied depending on the specific procedure, including the size and shape of the corresponding end of the endovascular implant.

The bend 14b may also be pre-formed in the wire 14 forming part of the apparatus 10 prior to positioning in the sheath 12, as noted. Alternatively, a shape memory material (e.g., Nitinol) may be used to allow the bend 14b and the shaped extension 14c to be formed in situ. In other words, the wire 14 may initially be linear and, as a result of activation of shape memory, such as based on a change in environmental condition (e.g., temperature), the bend 14b may take shape in a pre-determined manner (and likewise may be caused to disappear in like manner).

In summary, an apparatus 10 is for positioning an endovascular implant, such as a stent 18, adjacent a junction J including a branch B from a vessel V in a highly repeatable and reliable manner. A sheath 12 includes first and second lumens 12a, 12b, the first lumen 12a adapted for receiving an at least partially radiopaque wire 14, which may be smaller in diameter than the second lumen 12b for adapted for receiving a delivery device 16, such as a catheter 20 for positioning the endovascular implant within the branch B. The radiopaque wire 14 includes a preshaped portion 14b, 14c for positioning adjacent the junction J for use in guiding the endovascular implant into position within the branch B.

Summarizing, this disclosure may be considered to relate to the following items:
1. An apparatus for positioning an endovascular implant adjacent a junction including a branch from a vessel, comprising:
   a sheath including a first lumen and a second lumen, the first lumen adapted for receiving a catheter adapted for positioning the endovascular implant within the branch; and
   an at least partially radiopaque wire located in the second lumen, the at least partially radiopaque wire including a pre-shaped portion for positioning at the junction for use in guiding the endovascular implant into position within the branch.
2. The apparatus of item 1, wherein the first lumen has a larger diameter than the second lumen.
3. The apparatus of item 1 or item 2, wherein the at least partially radiopaque wire comprises metal.
4. The apparatus of any of items 1-3, wherein the at least partially radiopaque wire comprises a shape memory material.
5. The apparatus of any of items 1-4, wherein an end portion of the radiopaque wire distal of the pre-shaped portion is adapted for engaging an adjacent wall of the vessel at the branch.
6. The apparatus of any of items 1-5, wherein an opening of the second lumen is at a distal end of the sheath.
7. The apparatus of any of items 1-5, wherein an opening of the second lumen is proximal of a distal end of the sheath.
8. The apparatus of any of items 1-7, wherein the pre-shaped portion comprises a bend, such as one having a U-shape or one with one or more, such as two, curved portions, having a width and a depth, in the at least partially radiopaque wire proximal of a distal end of the at least partially radiopaque wire.
9. The apparatus of item 8, wherein the bend is adapted to engage an end of the endovascular implant.
10. An apparatus for positioning an endovascular implant adjacent a junction including a branch from a vessel, comprising:
   a sheath including a first lumen and a second lumen;
   a catheter at least partially positioned in the first lumen, the catheter adapted for positioning the endovascular implant within the branch; and
   an at least partially radiopaque wire located in the second lumen, the at least partially radiopaque wire including a pre-shaped portion for positioning at the junction for use in guiding the endovascular implant into position within the branch.
11. The apparatus of item 10, wherein the first lumen has a larger diameter than the second lumen.
12. The apparatus of item 10 or item 11, wherein the at least partially radiopaque wire comprises metal.
13. The apparatus of any of items 10-12, wherein the at least partially radiopaque wire comprises a shape memory material.
14. The apparatus of any of items 10-13, wherein an extension of the at least partially radiopaque wire is adapted for engaging an adjacent wall of the vessel at the branch.
15. The apparatus of any of items 10-14, wherein an opening of the second lumen is at a distal end of the sheath.
16. The apparatus of any of items 10-14, wherein an opening of the second lumen is proximal of a distal end of the sheath.
17. The apparatus of any of items 10-16, wherein the pre-shaped portion comprises a bend in the at least partially radiopaque wire proximal of a distal end of the at least partially radiopaque wire.
18. The apparatus of item 17, wherein the bend is adapted to engage an end of the endovascular implant.
19. An apparatus for positioning an endovascular implant adjacent a junction including a branch from a vessel using a sheath including a first lumen and a second lumen, the first lumen adapted for receiving a catheter adapted for positioning the endovascular implant within the branch, comprising:
   an at least partially radiopaque wire adapted to be located in the second lumen, the at least partially radiopaque wire including a preshaped portion adapted for positioning at the junction for use in guiding the endovascular implant into position within the branch.
20. The apparatus of item 19, wherein the at least partially radiopaque wire comprises metal.
21. The apparatus of item 19 or item 20, wherein the at least partially radiopaque wire comprises a shape memory material.
22. The apparatus of any of items 19-21, wherein an extension of the at least partially radiopaque wire is adapted for engaging an adjacent wall of the vessel at the branch.
23. The apparatus of any of items 19-22, wherein the pre-shaped portion comprises a bend in the at least partially radiopaque wire proximal of a distal end of the at least partially radiopaque wire.
24. The apparatus of item 23, wherein the bend is adapted to engage an end of the endovascular implant.
25. A method of positioning an endovascular implant in a branch from a vessel, comprising:
   providing a sheath including a first lumen; and
   extending an at least partially radiopaque wire from the first lumen to locate a pre-shaped portion of the at least partially radiopaque wire adjacent the branch for use in guiding the endovascular implant into position within the branch.
26. The method of item 25, further including the step of providing the sheath with a second lumen, and advancing a catheter including the endovascular implant via the second lumen into the branch.

As used herein, the following terms have the following meanings:

“A”, “an”, and “the” as used herein refers to both singular and plural referents unless the context clearly dictates otherwise. By way of example, “a compartment” refers to one or more than one compartment.

“About,” “substantially,” or “approximately,” as used herein referring to a measurable value, such as a parameter, an amount, a temporal duration, and the like, is meant to encompass variations of +/−20% or less, preferably +/−10% or less, more preferably +/−5% or less, even more preferably +/−1% or less, and still more preferably +/−0.1% or less of and from the specified value, in so far such variations are appropriate to perform in the disclosed invention. However, it is to be understood that the value to which the modifier “about” refers is itself also specifically disclosed.

“Comprise”, “comprising”, and “comprises” and “comprised of” as used herein are synonymous with “include”, “including”, “includes” or “contain”, “containing”, “contains” and are inclusive or open-ended terms that specifies the presence of what follows, e.g., component or the like does not exclude or preclude the presence of additional, non-recited components, features, element, members, steps, known in the art or disclosed therein.

Although the invention has been described in conjunction with specific embodiments, many alternatives, modifications, and variations will be apparent to those skilled in the art. Accordingly, it embraces all such alternatives, modifications, and variations that fall within the spirit and scope of the appended claims. It is to be fully understood that certain aspects, characteristics, and features, of the invention, which are, for clarity, illustratively described and presented in the context or format of a plurality of separate embodiments, may also be illustratively described and presented in any suitable combination or sub-combination in the context or format of a single embodiment. Conversely, various aspects, characteristics, and features, of the invention which are illustratively described and presented in combination or sub-combination in the context or format of a single embodiment may also be illustratively described and presented in the context or format of a plurality of separate embodiments.

1. An apparatus for positioning an endovascular implant adjacent a junction including a branch from a vessel, comprising:
a sheath including a first lumen and a second lumen, the first lumen adapted for receiving a catheter adapted for positioning the endovascular implant within the branch; and
an at least partially radiopaque wire located in the second lumen, the at least partially radiopaque wire including a pre-shaped portion for positioning at the junction for use in guiding the endovascular implant into position within the branch.

2. The apparatus of claim 1, wherein the first lumen has a larger diameter than the second lumen.

3. (canceled)

4. The apparatus of claim 1, wherein the at least partially radiopaque wire comprises a shape memory material.

5. The apparatus of claim 1, wherein an extension of the at least partially radiopaque wire is adapted for engaging a wall adjacent the branch.

6. The apparatus of claim 1, wherein an opening of the second lumen is at a distal end of the sheath.

7. The apparatus of claim 1, wherein an opening of the second lumen is proximal of a distal end of the sheath.

8. The apparatus of claim 1, wherein the pre-shaped portion comprises a U-shaped bend with two curved portions having a depth and a width, in the at least partially radiopaque wire proximal of a distal end of the at least partially radiopaque wire.

9. The apparatus of claim 8, wherein the bend is adapted to engage an end of the endovascular implant.

10. An apparatus for positioning an endovascular implant adjacent a junction including a branch from a vessel, comprising:
a sheath including a first lumen and a second lumen;
a catheter at least partially positioned in the first lumen, the catheter adapted for positioning the endovascular implant within the branch; and
an at least partially radiopaque wire located in the second lumen, the at least partially radiopaque wire including a pre-shaped portion for positioning at the junction for use in guiding the endovascular implant into position within the branch.

11. The apparatus of claim 10, wherein the first lumen has a larger diameter than the second lumen.

12. (canceled)

13. The apparatus of claim 10, wherein the at least partially radiopaque wire comprises a shape memory material.

14. The apparatus of claim 10, wherein an extension of the at least partially radiopaque wire is adapted for engaging an adjacent wall of the vessel at the branch.

15. The apparatus of claim 10, wherein an opening of the second lumen is at a distal end of the sheath.

16. The apparatus of claim 10, wherein an opening of the second lumen is proximal of a distal end of the sheath.

17. The apparatus of claim 10, wherein the pre-shaped portion comprises a bend in the at least partially radiopaque wire proximal of a distal end of the at least partially radiopaque wire.

18. The apparatus of claim 17, wherein the bend is adapted to engage an end of the endovascular implant.

19. An apparatus for positioning an endovascular implant adjacent a junction including a branch from a vessel using a sheath including a first lumen and a second lumen, the first lumen adapted for receiving a catheter adapted for positioning the endovascular implant within the branch, comprising:
an at least partially radiopaque wire adapted to be located in the second lumen, the at least partially radiopaque wire including a pre-shaped portion adapted for positioning at the junction for use in guiding the endovascular implant into position within the branch.

20.-21. (canceled)

22. The apparatus of claim 19, wherein an extension of the at least partially radiopaque wire is adapted for engaging an adjacent wall of the vessel at the branch.

23. The apparatus of claim 19, wherein the pre-shaped portion comprises a bend in the at least partially radiopaque wire proximal of a distal end of the at least partially radiopaque wire.

24. The apparatus of claim 23, wherein the bend is adapted to engage an end of the endovascular implant.

25.-27. (canceled)
